(19) United States
(12) Patent Application Publication
(10) Pub. No.: US 2009/0221697 A1
(43) Pub. Date: Sep. 3, 2009

(54) MACROLIDE CONJUGATES OF PYRROLIZINE AND INDOLIZINE COMPOUNDS AS INHIBITORS OF 5-LIPOOXYGENASE AND CYCLOOXYGENASE

(75) Inventors: Stefan Laufer, Tübingen (DE); Wolfgang Albrecht, Ulm (DE); Michael Burnet, Tuebingen (DE); Hans-Jürgen Gutke, München (DE)

Correspondence Address: Edwards Angell Palmer & Dodge LLP, P.O. Box 55874, Boston, MA, 02205, US

(73) Assignee: MERCKLE GMBH, Ulm (DE)

(21) Appl. No.: 11/996,801

(22) PCT Filed: Jul. 26, 2006
(86) PCT No.: PCT/EP06/07339

§ 371 Date: Jun. 27, 2008

Related U.S. Application Data
(60) Provisional application No. 60/702,775, filed on Jul. 26, 2005.

(51) Int. Cl. A61K 31/40; C07D 413/14; A61K 31/35; A61P 29/00

(52) U.S. Cl. 514/459; 540/467; 514/413

(57) ABSTRACT

The present invention relates to macrolide conjugates of pyrrolizine and indolizine derivatives with macrocyclic antibiotics and derivatives thereof. The macrolide conjugates are potent inhibitors of 5-lipoxygenase and cyclooxygenase and are therefore suitable to treat disorders of the rheumatic type and to prevent allergically induced diseases. The macrolide conjugates have significantly enhanced potency and efficacy.

The present invention relates to macrolide conjugates of pyrrolizine and indolizine compounds and to pharmaceutical compositions containing them.

Macrolides are macrocyclic lactones which are naturally derived and semi-synthetic compounds having a broad range of biological activities. Amongst the best-known of these biological activities is antibiotic activity which is achieved through binding to the bacterial ribosome. Macrolides having biological activity are for example disclosed in U.S. Pat. No. 3,478,018; U.S. Pat. No. 3,652,537; U.S. Pat. No. 4,328,334; U.S. Pat. No. 5,543,400; WO 95/09601; WO 02/32917; WO 02/50091; WO 02/087596; WO 03/42228; WO 03/077830; WO 04/052904; WO 04/101585; WO 04/101586; WO 04/101587; WO 04/101588; WO 04/101190; WO 04/106353; WO 04/101354; and EP 467 331 A.

In recent years macrocycles have found broader application as drug carriers in which an active substance is covalently but reversibly bonded to the macrocycle via a chemical bond, such as an ester bond to form a macrolide conjugate. Such conjugates are known with linker between the macrocycle and the active substance or without such linker, see for example WO 03/070173; WO 03/070174; and WO 03/070173. Conjugates with linker between the carrier and the drug are further known from WO 02/055531; WO 04/05313; WO 04/005309; and WO 04/094449.

The underlying rationale for this is the well-known property of many macrolide antibiotics to accumulate in many immunocompetent cells including neutrophils, monocytes, eosinophils, macrophage, alveolar macrophage, B and T-lymphocytes, NK cells, giant cells, Kupfer cells, glial cells, and similar target cells so that autoimmune diseases may be treated by using pro-drugs based on macrolides. Examples from several drug classes have been shown to benefit from conjugation to macrolide antibiotics or macrolide derived antibiotics. These drug classes include COX inhibitors, corticosteroids, cytostatics and IMPDH inhibitors. Azalide derived compounds seem to be especially suitable. It is generally desirable to employ non-antibiotic compounds to prevent bacterial resistance. Therefore modifications of the parent macrolide are desirable that will abolish antibacterial activity but keep the favourable properties with regard to cellular and gut uptake. Modifications will also affect cleavage kinetics meaning that modulation of drug half-life is feasible. On the other hand cellular uptake and other parameters will also be dependent on the nature of the drug coupled to the macrolide carrier molecule. The chemical structure of the drug is not a criterion for the uptake into the immune cells. Rather, similar molecules with similar properties can exhibit quite different uptake into immune cells, see WO 03/070174, page 58. This is in particular true for a group of pyrrolizine and indolizine compounds which are disclosed in U.S. Pat. No. 5,260,451; U.S. Pat. No. 5,939,415; and U.S. Pat. No. 5,958,943. One of said compounds is licofelone (ML 3000) which is a promising inhibitor of cyclooxygenase and 5-lipoxygenase and has the structural formula

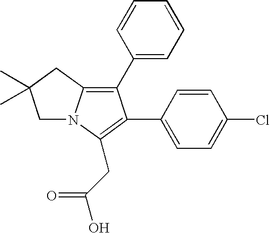

These compounds are highly water-insoluble and, moreover, unstable so that it is a problem to prepare suitable pharmaceutical dosage forms which are highly effective and stable.

The problem underlying the present invention is therefore to provide modified forms of the above-mentioned pyrrolizine compounds which have improved anti-inflammatory activity and which allow to obtain stable dosage forms.

It was now surprisingly found that this problem is solved by certain macrolide conjugates of said pyrrolizine and indolizine compounds.

### SUMMARY OF THE INVENTION

The present invention relates to macrolide conjugates of the following formula I

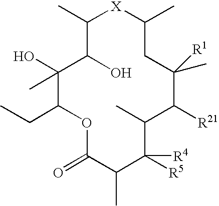

wherein

R¹ is hydroxy or C₁-C₄-alkoxy or

R¹ and R⁴ together with the carbon atoms to which they are attached form a tetrahydrofurane ring,

R²¹ is

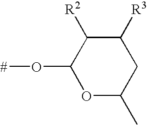

one of radicals R² and R³ is OR⁹ and the other is NR⁶R⁷;

R⁴ is OH, OR¹⁰ or

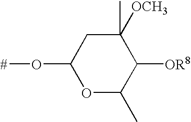

R⁵ is H or

R⁴ and R⁵ together with the carbon atom to which they are attached form a carbonyl group;

R⁶ and R⁷ which may be the same or different are C₁-C₄-alkyl or R⁹O—C₁-C₄-alkyl;

R⁸ is H or R¹⁰;

R⁹ is H or R¹⁰;

R¹⁰ is

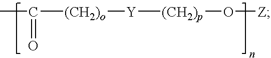

X is NR¹¹CH₂, CH₂NR¹¹, C═O or C═NOR²⁰;

R¹¹ is H or C₁-C₄-alkyl;

R²⁰ is H, R¹⁰ or —(CH₂)ₖ—Y—(CH₂)ₗ—Y—(CH₂)ₘ—CH₃;

Y is O or a bond;

k is 1 or 2;

l is 1, 2 or 3;

m is 0, 1 or 2;

n is 0 or 1;

o is 1, 2, or 3;

p is 1, 2, or 3;

Z is

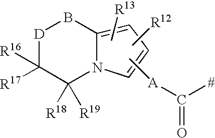

wherein

R¹² and R¹³ which may be the same or different are selected from:

phenyl which is optionally substituted with 1 or 2 halogen, hydroxy, C₁-C₄-alkoxy, phenoxy, C₁-C₄-alkyl or CF₃,
a 5- or 6-membered aromatic heterocyclic group containing 1, 2 or 3 heteroatoms selected from O, N, or S and which may be substituted with 1 or 2 halogen, C₁-C₄-alkyl or CF₃,
a benzofused 5- or 6-membered aromatic heterocyclic group containing 1, 2 or 3 heteroatoms selected from O, N, or S and which may be substituted with 1 or 2 halogen, C₁-C₄-alkyl or CF₃;

A is a bond or C₁-C₈-alkylene which can optionally be substituted by hydroxyl or C₁-C₄-alkoxy;

B is CR¹⁴R¹⁵ or C═O;

R¹⁴ and R¹⁵ which may be the same or different are H or C₁-C₄-alkyl or one of radicals R¹⁴ and R¹⁵ is H, C₁-C₄-alkyl, hydroxy-C₁-C₄-alkyl or C₁-C₄-alkoxy-C₁-C₄-alkyl and the other is OH, C₁-C₄-alkoxy or C₁-C₄-alkylcarbonyloxy;

D is a bond between B and the carbon atom carrying R¹⁶ and R¹⁷ or is CH₂;

R¹⁶ and R¹⁷ which may be the same or different are H, C₁-C₄-alkyl, hydroxy-C₁-C₄-alkyl or C₁-C₄-alkoxy-C₁-C₄-alkyl;

R¹⁸ and R¹⁹ which may be the same or different are H or C₁-C₄-alkyl or

two of radicals R⁶, R¹⁷, R¹⁸ and R¹⁹ form a double bond and the two others are H or C₁-C₄-alkyl,
and the pharmaceutically acceptable salts, solvates, hydrates and stereochemical isomers thereof.

Further, the invention relates to pharmaceutical compositions containing those macrolide conjugates of formula I which comprise at least one group Z and to the use thereof for the manufacture of pharmaceutical compositions for the treatment of disorders of the rheumatic type.

### DETAILED DESCRIPTION OF THE INVENTION

The term “alkyl” refers to a hydrocarbon chain that may be a straight chain or branched chain containing the indicated number of carbon atoms. Examples for such alkyl groups are methyl, ethyl, n-propyl, isopropyl, n-butyl, sec-butyl, iso-butyl and t-butyl.

The term “alkoxy” is an O-alkyl group in which the alkyl group is as defined above.

The term “halogen” means radicals of fluorine, chlorine, bromine or iodine, preferably fluorine or chlorine.

The term “alkylene” refers to straight chain or branched alkylene groups having the indicated number or carbon atoms. Preferred is C₁-C₄-alkylene, and in particular —CH₂—, —CH₂—CH₂—, —CH₂CH₂CH₂— and —CH(CH₃)CH₂—.

# indicates the bond where the group is attached.

Examples for 5-membered aromatic heterocyclic groups are thienyl, furyl, pyrrolyl, thiazolyl, isothiazolyl, oxazolyl, isoxazolyl, imidazolyl, pyrazolyl, thiadiazolyl, oxadiazolyl and triazolyl.

Examples for 6-membered aromatic heterocyclic groups are pyridinyl, pyrimidinyl, and triazinyl,

Examples for benzofused aromatic heterocyclic groups are benzothienyl, benzofuryl, indolyl and quinolinyl.

Preferred aromatic heterocyclic groups are thienyl, chlorothienyl, furyl, and benzofuryl.

The physiologically acceptable salts of the compounds of formula I are in particular acid addition salts. The acid addition salts may be formed with inorganic acids such as hydrochloric acid, sulphuric acid or phosphoric acid, or with organic acids such as tartaric acid, citric acid, maleic acid, fumaric acid, malic acid, mandelic acid, ascorbic acid, gluconic acid, methane sulfonic acid, toluene sulfonic acid etc.

The compounds of formula I contain asymmetric carbon atoms. They may therefore exist in the form of racemates, racemic mixtures, single enantiomers, diastereomers, diastereomeric mixtures or syn- and anti-isomers (in case X is C═NOR²⁰). All these forms are comprised by the present invention.

R¹ is preferably hydroxy or forms together with R⁴ and the carbon atoms to which they are attached a tetrahydrofurane ring.

A preferred embodiment are the compounds of formula I wherein R² is OR⁹ and R³ is NR⁶R⁷. In such case R² is preferably OR¹⁰ and R³ is NR⁶R⁷, wherein R⁶ and R⁷ are C₁-C₄-alkyl, in particular methyl. Alternatively, R² is hydroxy and R³ is NR⁶R⁷, wherein R⁶ is C₁-C₄-alkyl and R⁷ is R¹⁰O—C₁-C₄-alkyl.

A further preferred embodiment are compounds of formula I, wherein R² is NR⁶R⁷ and R³ is OR⁹. Preferably, R³ is OR¹⁰ and R⁶ and R⁷ are C₁-C₄-alkyl. Alternatively, R³ is hydroxy and R⁶ is C₁-C₄-alkyl and R⁷ is R¹⁰O—C₁-C₄-alkyl.

R²¹ is preferably

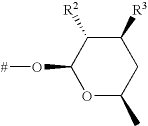

R⁴ is preferably hydroxy or a residue of the formula

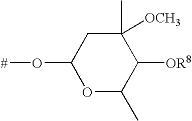

and in particular of the formula

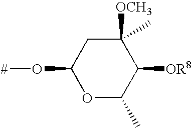

wherein R⁸ is H or R¹⁰.

Alternatively, R⁴ and R⁵ form together with the carbon atom to which they are attached a carbonyl group.

X is preferably
a) NR¹¹CH₂ which results in a compound of the following formula

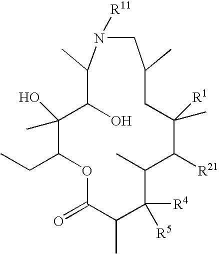

wherein R¹¹ is C₁-C₄-alkyl and R¹, R²¹, R⁴ and R⁵ are as defined above;
b) C═NO(CH₂)ₖY(CH₂)ₗY(CH₂)ₘ—CH₃ wherein Y, k, l and m are as defined above. Preferably, Y, k, l and m are selected such that they give the following group: C═NOCH₂O(CH₂)₂O—CH₃;
c) C═NOR¹⁰ wherein R¹⁰ is as defined above.

The macrolide conjugates of formula I include the residue of a pyrrolizine or indolizine drug which contains a carboxyl group. Z is the residue of said drug after removal of the hydroxy group of the carboxyl group. Z is covalently bonded either directly or via a linker to the macrolide residue. If the drug residue is directly bonded to the macrolide residue, then R¹⁰ is Z. If it is bonded via a linker, R¹⁰ is preferably —[—CO—(CH₂)ₒ—Y—(CH₂)ₚ—O-]-Z wherein Y, z, o and p are as defined above. Preferably, Y is a bond and o+p=2 or 3.

The pyrrolizine and indolizine residue Z has the formula

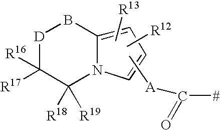

wherein A, B, D, R¹² to R¹⁹ are as defined above. A is preferably methylene or ethylene.

B is preferably CH₂. D is preferably a bond between B and the carbon atom carrying R¹⁶ and R¹⁷.

R¹⁶ and R¹⁷ are preferably and independently from each other hydrogen or C₁-C₄-alkyl or two of R¹⁶, R¹⁷, R¹⁸ and R¹⁹ form a double bond and the two others are H or C₁-C₄-alkyl. R¹⁸ and R¹⁹ preferably are hydrogen.

R¹² and R¹³ which may be the same or different are preferably phenyl, thienyl, furyl, pyrrolyl, imidazolyl, thiadiazolyl, oxazolyl, pyridinyl, pyrimidyl, benzofuryl or quinolyl and may optionally be substituted with one or two halogen or CF₃. The preferred halogen substituent is F or Cl. R¹² and R¹³ are particularly preferred phenyl, halogen-substituted phenyl, thienyl, halogen-substituted thienyl or benzofuryl.

According to a particularly preferred embodiment Z has the formula

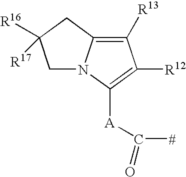

wherein A is a bond or C₁-C₈-alkylene, in particular CH₂ or CH₂CH₂,

R¹² is phenyl, thienyl or benzofuryl which is optionally substituted with halogen, in particular chlorophenyl, chlorothienyl or benzofuryl,

R¹³ is phenyl and

R¹⁶ and R¹⁷ are hydrogen or C₁-C₄-alkyl.

In a furthermore preferred embodiment R¹⁶ and R¹⁷ are methyl, R¹² is 4-chlorophenyl, 5-chlorothien-2-yl or benzofur-2-yl and the most preferred residue Z is

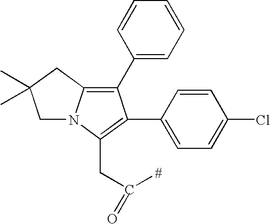

Particularly preferred macrolide conjugates have the following formulae:

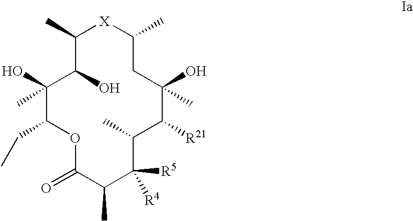

In formula Ia R²¹, R⁴, R⁵ and X are as defined above.

Particularly preferred embodiments are the macrolide conjugates of formulae Iaa to Iaf:

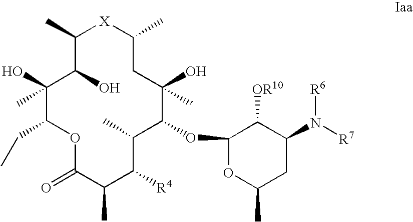

wherein R⁶ and R⁷ which may be the same or different are C₁-C₄-alkyl;

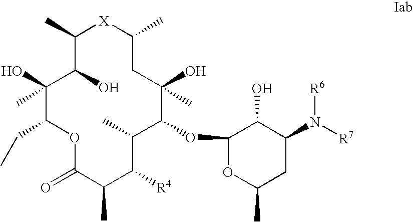

wherein R⁶ is C₁-C₄-alkyl and R⁷ is hydroxy-C₁-C₄-alkyl or R¹⁰O—C₁-C₄-alkyl;

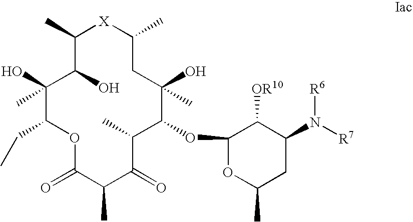

wherein R⁶ and R⁷ which may be the same or different are C₁-C₄-alkyl;

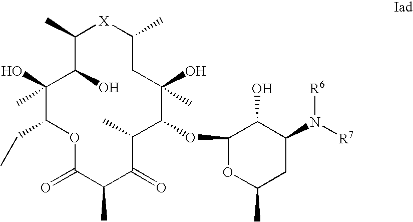

wherein R⁶ is C₁-C₄-alkyl and R⁷ is hydroxy-C₁-C₄-alkyl or R¹⁰ O—C₁-C₄-alkyl;

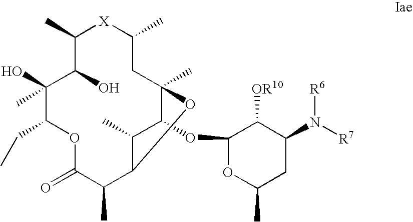

wherein R⁶ and R⁷ which may be the same or different are C₁-C₄-alkyl;

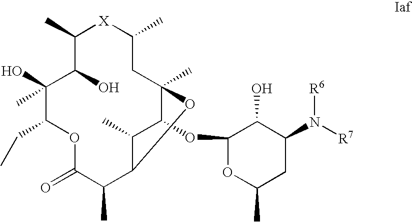

wherein R⁶ is C₁-C₄-alkyl and R⁷ is hydroxy-C₁-C₄-alkyl or R¹⁰O—C₁-C₄-alkyl; and wherein in formulae Iaa to Iaf R⁴ is hydroxy or

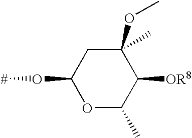

and X, R⁸ and R¹⁰ are as defined above.

The preparation of the starting materials for the preparation is disclosed in WO 03/070173, WO 03/070174 and WO 2004/005309 or can be done in an analogous manner. Also, the macrolide conjugates of the present invention can be prepared in analogy to the methods disclosed in WO 03/070173, WO 03/070174 and WO 2004/005309. The preparation of the macrolide conjugates of the present invention starts out from azithromycin, erythromycin or roxithromycin. The compounds of formula I wherein X is NR¹¹CH₂ can be prepared from azithromycin by subjecting it to the conversions which are shown in reaction schemes 1 and 2.

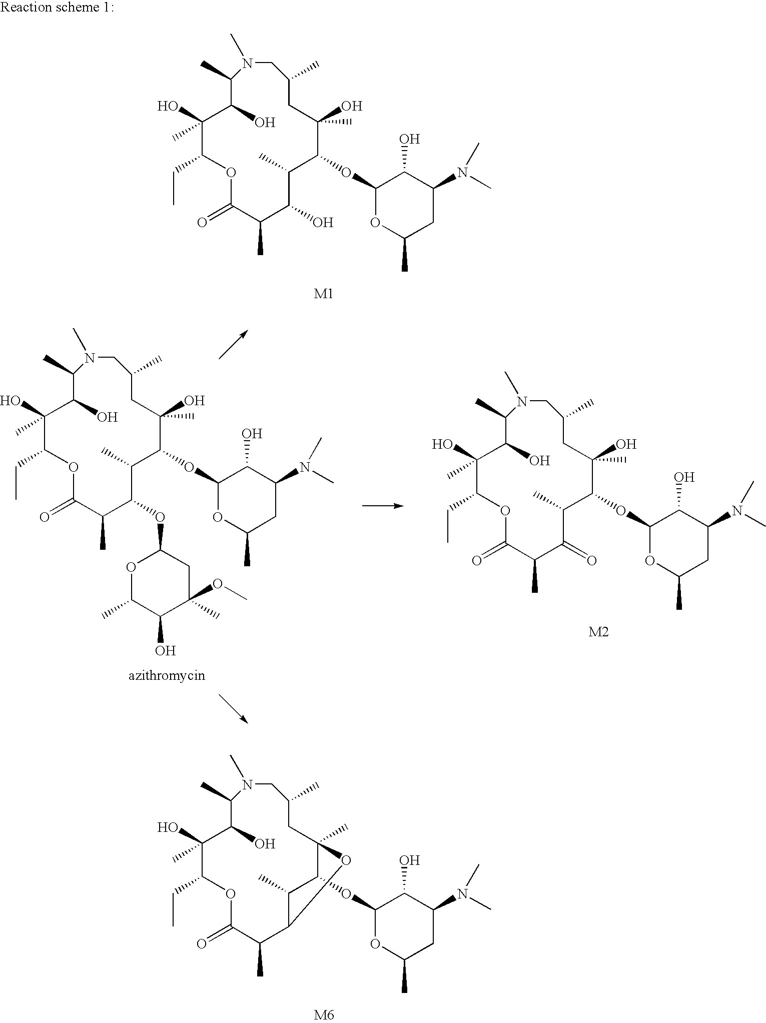

In order to obtain the intermediate compound M1, azithromycin is treated with a diluted mineral acid such as hydrochloric acid or sulfuric acid (confer example 2 of WO 02/070174). The acidic hydrolysis gives the decladinosylated product in high yield which can be used as starting material for the introduction of various functional groups or which can be used for directly conjugating a drug to the 2′-position.

Azithromycin can also be directly converted to intermediate M2 which is the starting material for the preparation of compounds of formula I wherein R⁴ and R⁵ together with the carbon atom to which they are bonded form a carbonyl group. To obtain M2, N-chlorosuccinimide is contacted with dimethylsulfide in a chlorinated hydrocarbon solvent such as dichloromethane. The obtained precipitate is then reacted with azithromycin, see example 3 of WO 03/070174.

Azithromycin can also be heated in dimethylformamide in the presence of sodium azide to give intermediate M6.

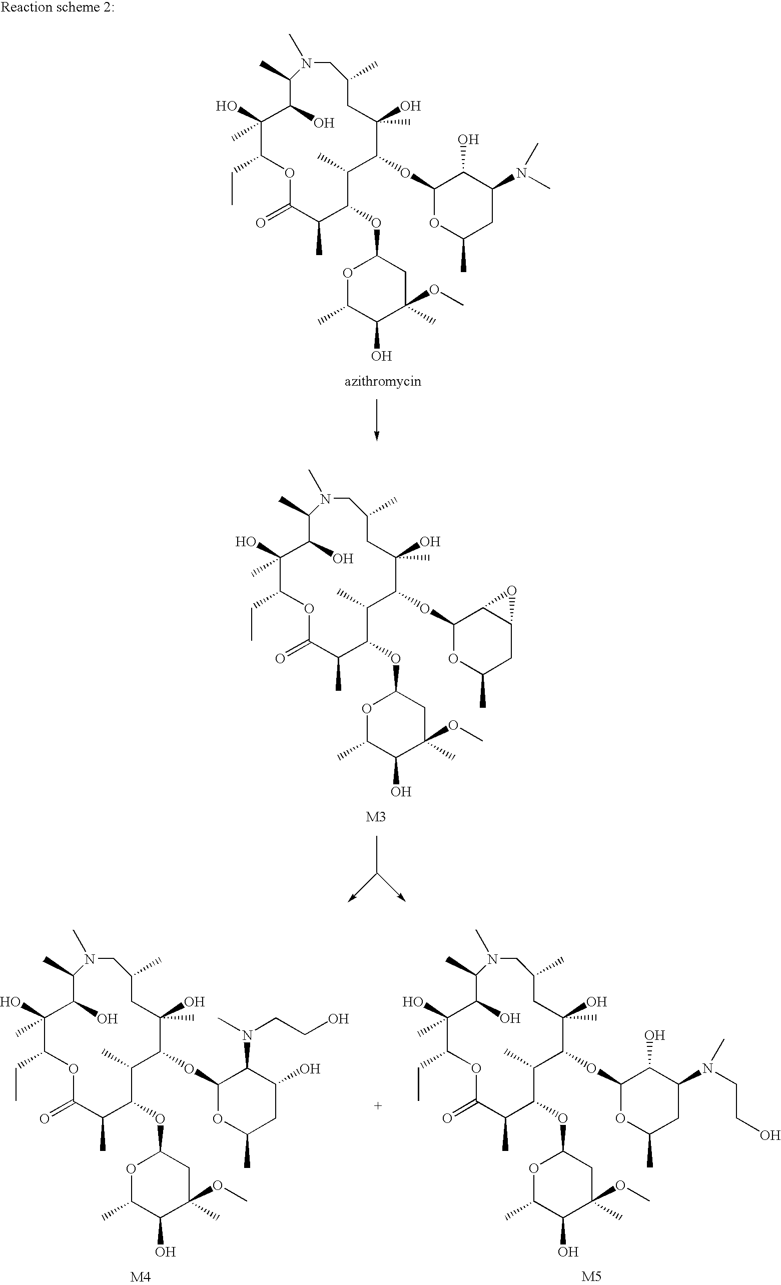

As shown in reaction scheme 2, azithromycin is converted to intermediate M3 by reaction with epichlorohydrine. The oxirane ring in M3 may then be opened by a variety of nucleophils, in particular by secondary amines and aminoalcohols. In reaction scheme 2 this reaction is illustrated with hydroxyethyl methylamine to give the regioisomers M4 and M5.

The compounds of formula I wherein X is C═O or C═NOR²⁰ can be obtained from erythromycin A or roxithromycin of the formula

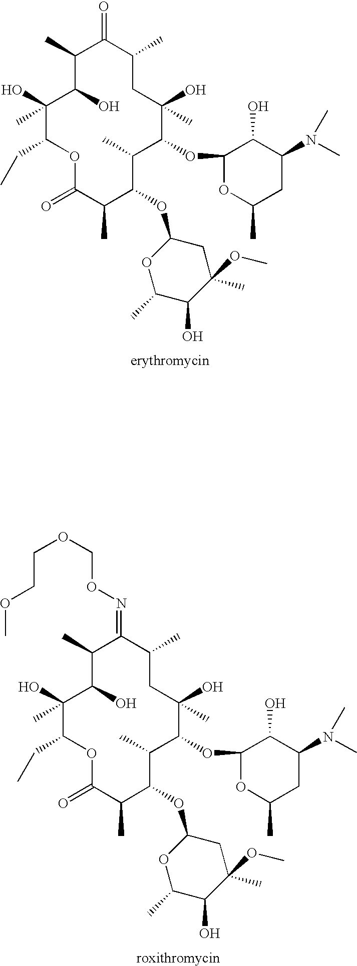

Erythromycin A can be modified by converting the keto group to the corresponding oxime ethers (X is C═NOR²⁰) in a conventional manner by reacting with hydroxylamine and etherifying the hydroxy group. Alternatively roxithromycin can be used as starting material. Erythromycin, the oxime ethers thereof and roxithromycin can be subjected to the same reactions as illustrated in reaction schemes 1 and 2 to give suitable starting materials for the introduction of the drug residue Z.

Said drug residue Z can be introduced by a coupling reaction (esterification) between the drug carboxylic acid and an alcohol group of the above described starting materials. This coupling reaction in general comprises an activation step for the drug carboxylic acid. The activation can be conveniently effected by dicyclohexylcarbodiimide, N,N′-carbonyldiimidazole or 1-ethyl-3-(3-dimethylaminopropyl)carbodiimide in the presence of said intermediate. The selectivity of the coupling reaction depends on the starting material used and on the esterification catalyst. The reactions may take place at temperatures from −20° C. to 50° C. It is most convenient to start at ice bath temperature and to let the reaction finish at ambient temperature. Preferably, the reaction is carried out in an inert organic solvent such as an ether, like tetrahydrofurane, dioxane or dimethoxyethane, ester such as ethylacetate, halogenated hydrocarbon, such as methylene chloride or acetonitrile. Workup of the reaction mixture and purification of the macrolide conjugate is carried out in a conventional manner. Purification is preferably performed by column chromatography on silica gel employing a slightly basic eluent system containing ammonia or a volatile amine.

The compounds of the present invention are stable and highly active in the treatment of disorders of the rheumatic type and for the prevention of allergically induced disorders. They are thus effective anti-inflammatories, analgesics, antipyretics, antiallergics and broncholytics or have antibronchoconstrictor activity. They can therefore be used for thrombosis prophylaxis and for the prophylaxis of anaphylactic and septic shock as well as for the treatment of dermatological disorders of allergic and non-allergic genesis, such as psoriasis, urticaria, acute and chronic exanthema. In particular, they can be used for treating arthritis, especially rheumatoid arthritis.

The compounds have increased chemical stability and improved bioavailability as compared to the unconjugated drug and can be administered parenterally.

The compounds according to the invention can either be administered as individual therapeutic active compounds or as mixtures with other therapeutic active compounds. They can be administered as such, but in general they are administered in the form of pharmaceutical compositions, i.e. as mixtures of the active compounds with pharmaceutically acceptable excipients, in particular vehicles or diluents and/or additives. The compounds or compositions can be administered enterally, e.g. orally or rectally, or parenterally, e.g. subcutaneously, intravenously or intramuscularly, but they are preferably given in oral dosage forms. Due to the stability of the present compounds also topical dosage forms can be provided.

The nature of the pharmaceutical composition and of the pharmaceutical carrier or diluent depends on the desired manner of administration. Oral compositions can be present, for example, as tablets or capsules and can contain customary excipients, such as binding agents (e.g. syrup, acacia, gelatin, sorbitol, tragacanth or polyvinylpyrrolidone), fillers (e.g. lactose, sugar, maize starch, calcium phosphate, sorbitol or glycine), lubricants (e.g. magnesium stearate, talc, polyethylene glycol or silica), disintegrating agents (e.g. starch) or wetting agents (e.g. sodium laurylsulphate). Oral liquid preparations can be present in the form of aqueous or oily suspensions, solutions, emulsions, syrups, elixirs or sprays etc. or can be present as dry powders for reconstitution with water or another suitable carrier. Liquid preparations of this type can contain customary additives, for example suspending agents, flavourings, diluents or emulsifiers. For parenteral administration, solutions or suspensions with customary pharmaceutical carriers can be employed.

The use of compounds according to the invention in the course of treatment comprises administering an effective amount of one or more compounds, as a rule formulated corresponding to pharmaceutical and veterinary practice to the individual to be treated, preferably a mammal, in particular a human, agricultural animal or pet. Whether such a treatment is indicated and in which form it has to be carried out, depends on the individual case and is subject to medical assessment (diagnosis) of developing the present signs, symptoms and/or dysfunctions, risks, specific signs, symptoms and/or dysfunctions, and includes further factors.

As a rule, the treatment is carried out by administration one or more times daily, if appropriate together or alternately with other active compounds or active compound-containing preparations, so that a daily dose of approximately 0.1 mg to approximately 1000 mg and in particular 0.5 mg to approximately 100 mg per kg of body weight is administered to an individual to be treated.

The following examples illustrate the invention without restricting it.

### EXAMPLES

Solvents and reagents were commercial grade and were generally used without further purification. If dry solvents were required they were dried with and kept over molecular sieve 4 Å. TLC analysis was performed on silica gel 60 plates on aluminium foil, Merck Darmstadt. Visualisation was made by UV using quenching and staining with anisealdehyde reagent. Column chromatography was performed in open glass columns, on silica gel, Merck Darmstadt.

### ABBREVIATIONS

TLC: Thin layer chromatography
MS: Mass spectroscopy

### DMF: Dimethylformamide

### DCC: Dicyclohexylcarbodiimide

### THF: Tetrahydrofuran

### CDI: N,N′-Carbonyldiimidazole

EDCI: 1-Ethyl-3-(3-dimethylaminopropyl)carbodiimide

### Example 1

### M4 and M5

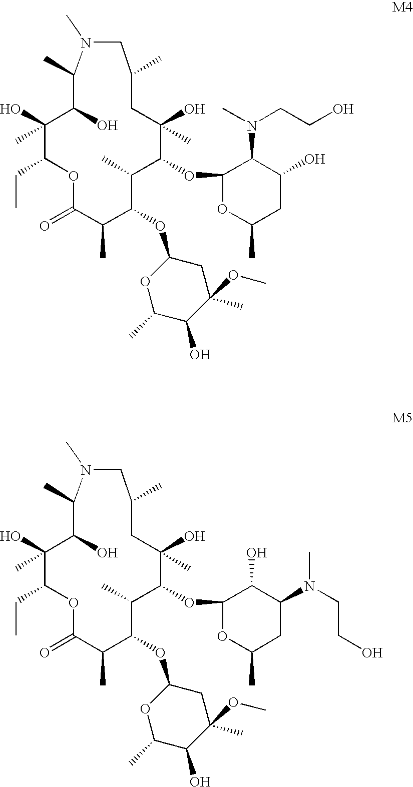

To a solution of 4.5 g of 2-ethyl-3,4,10-trihydroxy-13-(5-hydroxy-4-methoxy-4,6-dimethyl-tetrahydro-pyran-2-yloxy)-3,5,6,8,10,12,14-heptamethyl-11-(4-methyl-3,7-dioxa-bicyclo[4.1.0]hept-2-yloxy)-1-oxa-6-aza-cyclopentadecan-15-one (M3), prepared as described in WO 03/070174, in 20 ml of DMF at 80° C. 3 g of 2-hydroxyethyl methylamine were added and the heating was continued for 20 h. After cooling most of the DMF was evaporated in vacuum. The residue was taken up in 80 ml of ethyl acetate. The solution was washed with water and brine, dried (Na₂SO₄) and the solvent was evaporated in vacuum. The residue was chromatographed on silica gel, elution with chloroform:isopropanol:ammonia (7 M in methanol) 20:1:1 to yield 1.8 g of compound M5 and 2.5 g of compound M4.

### Example 2

### M6

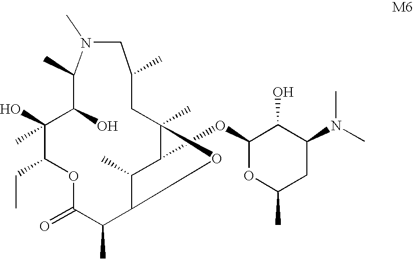

A solution of 12 g of azithromycin in 140 ml of DMF was heated to reflux in the presence of 5 g of powdered sodium azide for 48 h. After this period most of the solvent was removed in vacuum and the residue poured into water. The mixture was extracted with ethyl acetate. The combined organic extracts were washed with brine, dried (Na₂SO₄) and concentrated in vacuum. The residue was chromatographed on silica gel, elution with chloroform:isopropanol:ammonia (7 M in methanol) to yield 3.6 g of M6 a s slightly brownish solid.

### Example 3

### Compound 1

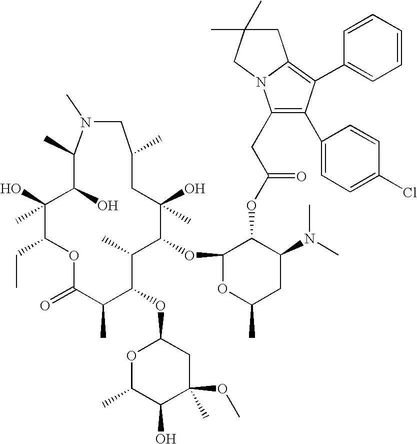

A stirred solution of 1.5 g of azithromycin in 9 ml of dry THF was cooled to 0° C. and 1.1 g of licofelone was added, followed by 0.59 g of DCC. After 1 h the mixture was allowed to come to ambient temperature and was stirred for another 12 h. The mixture was filtered and the filtrate concentrated in vacuum. The residue was taken up in toluene and chromatographed on silica gel (column 15 cm×2.5 cm, elution with chloroform:isopropanol:ammonia (7 M in methanol) 40:1:1 to yield the desired product.

### Example 4

### Compound 2

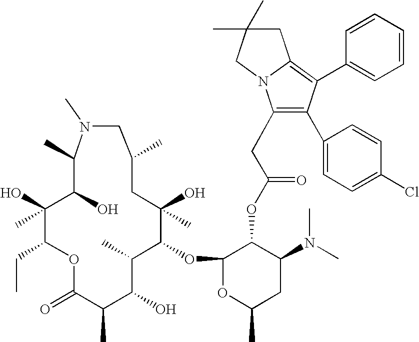

0.5 g of compound 1 were dissolved in 1 M HCl at ambient temperature. After 3 h the mixture was extracted with diethyl ether. The organic phase was discarded and the ice-cooled aqueous phase was treated with potassium carbonate to adjust the pH to about 10. The mixture was extracted with dichloromethane. The combined organic extracts were washed with brine, dried (Na₂SO₄) and concentrated in vacuum to yield the desired product, identical by MS and TLC with the product obtained as described in example 7.

### Example 5

### Compound 3

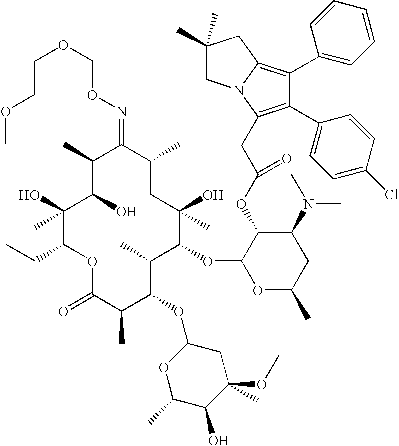

A suspension of 1.2 g of roxithromycin in 6 ml of THF was cooled to 0° C. and 820 mg of licofelone was added under rapid stirring, followed by 430 mg of DCC. The mixture was kept at the same temperature for 30 min and was then allowed to come to ambient temperature. After another 16 h the mixture was filtered and the filtrate concentrated in vacuum. The residue was chromatographed on silica gel, elution with chloroform:isopropanol:ammonia (7 M in methanol) 25:1:1 to yield the desired product, 1.5 g, as colorless solid.

### Example 6

### Compound 4

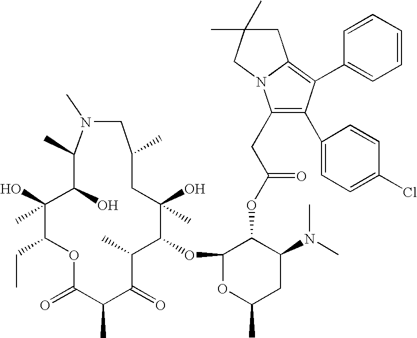

In a round bottom flask 5.9 g of M2 were suspended in 20 ml of dry THF. The mixture was cooled to 0-5° C. in an ice bath and 5.2 g of licofelone (ML3000) were added, immediately followed by 2.8 g of DCC. The mixture was stirred rapidly for 0.5 h at the same temperature and then the ice bath was removed. The mixture was stirred for another 12 h at ambient temperature and then filtered. The residue was washed with 20 ml of dichloromethane. All volatiles were removed in vacuum. The residue was taken up in 5 ml of toluene and 5 ml of dichloromethane and transferred on a column of silica gel, 25 cm×4.5 cm, packed as a slurry in dichloromethane:isopropanol:ammonia (7 M in methanol) 100:1:1. As eluent 400 ml of dichloromethane:isopropanol:ammonia (7 M in methanol) 60:1:1, changing to dichloromethane:isopropanol:ammonia (7 M in methanol) 30:1:1 were used. The collected fractions containing the product were evaporated at reduced pressure with a bath temperature not exceeding 30° C. to yield 6.8 g of the desired product.

### Example 7

### Compound 2

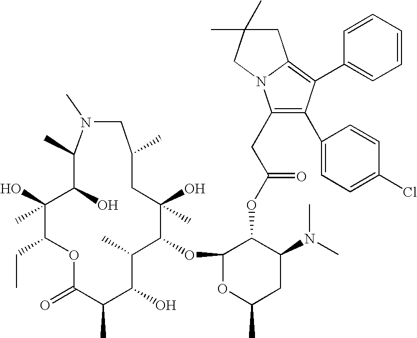

20 ml of dry THF were placed in a round bottom flask in which 5.9 g of M1 were suspended. The mixture was cooled to 0-5° C. in an ice bath and 5.2 g of licofelone were added, immediately followed by 2.8 g of DCC. The mixture was stirred for 0.5 h at the same temperature and then the ice bath was removed. The mixture was stirred for another 12 h at ambient temperature and then filtered. The residue was washed with 20 ml of dichloromethane. All volatiles were removed in vacuum. The residue was taken up in 5 ml of toluene and 5 ml of dichloromethane and transferred on a column of silica gel, 35 cm×4.5 cm, packed as a slurry in dichloromethane:isopropanol:ammonia (7 M in methanol) 100:1:1. As eluent 600 ml of dichloromethane:isopropanol:ammonia (7 M in methanol) 60:1:1, changing to dichloromethane:isopropanol:ammonia (7 M in methanol) 30:1:1 were used. The collected fractions containing the product were evaporated at reduced pressure with a bath temperature not exceeding 30° C. to yield 6.8 g of the desired product. Rf=0.28 (in chloroform:isopropanol:ammonia=30:1:1). MS: 476.7 (M+2H⁺).

### Example 8

### Compound 5

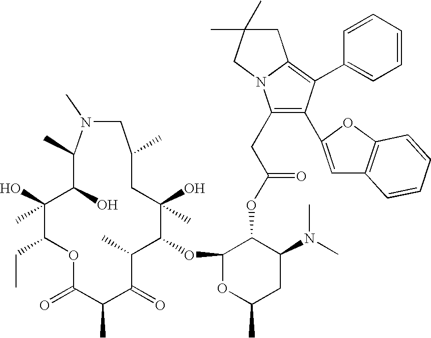

800 mg of (2-benzofuran-2-yl-6,6-dimethyl-1-phenyl-6,7-dihydro-5H-pyrrolizin-3-yl)-acetic acid) in 5 ml of THF were treated with 350 mg of CDI at ambient temperature. After 5 min 700 mg of M2 were added and the mixture was stirred for 24 h at ambient temperature. The mixture was concentrated in vacuum and the residue chromatographed on silica gel, elution with chloroform:isopropanol:ammonia (7 M in methanol) 40:1:1 to yield the desired product, 0.75 g slightly brownish solid, R_{f}=0.63 (in chloroform:isopropanol:ammonia=30:1:1).

### Example 9

### Compound 8

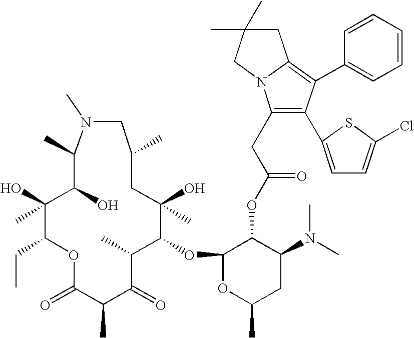

A solution of 1.1 g of M2 in 7 ml of dry THF was cooled to 0° C. 800 mg of [2-(5-chloro-thiophen-2-yl)-6,6-dimethyl-1-phenyl-6,7-dihydro-5H-pyrrolizin-3-yl]-acetic acid) were added under stirring, followed by 470 mg of DCC. The mixture was stirred rapidly at the same temperature for 1 h and was then allowed to come to ambient temperature. After 12 h the mixture was filtered and the residue concentrated in vacuum. The residue was chromatographed on silica gel, elution with chloroform:isopropanol:ammonia (7 M in methanol) 40:1:1 to yield 1.3 g of the desired product R_{f}=0.69 (in chloroform:isopropanol:ammonia=30:1:1). MS: 475.7 (M+2H⁺).

### Example 10

### Compound 7

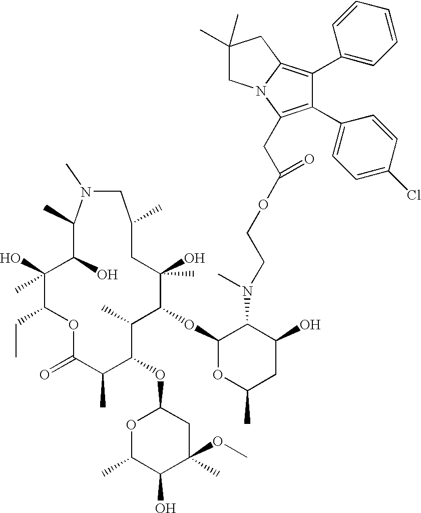

A solution of 670 mg of M4 in 4 ml of dry THF was cooled to 0° C. and 600 mg of licofelone were added under stirring, followed by 290 mg of DCC. After 30 min the mixture was allowed to come to ambient temperature and stirred for another 10 h. The mixture was filtered and the residue concentrated in vacuum, chromatographed on silica gel, elution with chloroform:isopropanol:ammonia (7 M in methanol) 40:1:1 to yield 0.75 g of the desired product. R_{f}=0.67 (chloroform:isopropanol:ammonia=30:1:1). MS: 570.7 (M+2H⁺).

### Example 11

### Compound 8

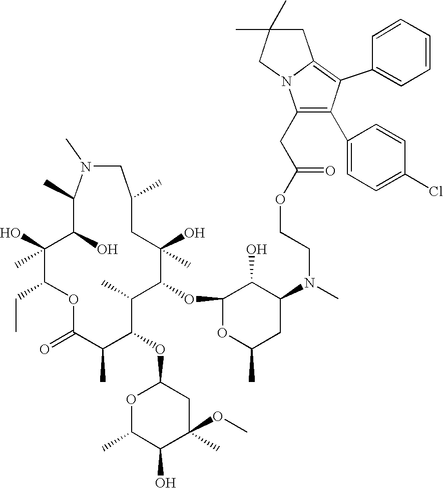

A solution of 430 mg of M5 in 4 ml of dry THF was cooled to 0° C. and 600 mg of licofelone were added under stirring, followed by 290 mg of DCC. After 30 min the mixture was allowed to come to ambient temperature and stirred for another 10 h. The mixture was filtered and the residue concentrated in vacuum, chromatographed on silica gel, elution with chloroform:isopropanol:ammonia (7 M in methanol) 40:1:1 to yield 0.75 g of the desired product, Rf=0.41 (chloroform:isopropanol:ammonia=30:1:1). MS: 570.7 (M+2H⁺).

### Example 12

### Compound 9

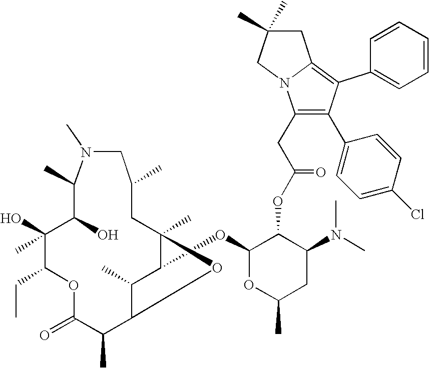

A solution of 1.5 g of M6 in 6 ml of THF was cooled to 0° C. and 1.4 g of licofelone were added under rapid stirring, followed by 770 mg of DCC. The mixture was kept at the same temperature for 30 min and then allowed to come to ambient temperature. After another 10 h the mixture was filtered and the filtrate concentrated in vacuum. The residue was chromatographed on silica gel, elution with chloroform:isopropanol:ammonia (7 M in methanol) 40:1:1 to yield 1.7 g of the desired product, R_{f}=0.40 (chloroform:isopropanol:ammonia=30:1:1). MS: 467.7 (M+2H⁺).

### Example 13

### Compound 10

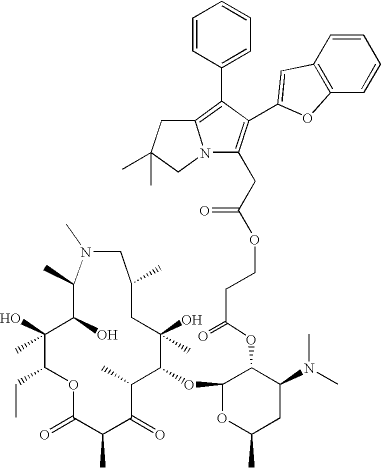

A solution of 400 mg of 2-benzofuran-2-yl-6,6-dimethyl-1-phenyl-6,7-dihydro-5H-pyrrolizin-3-yl)-acetic acid in 7 ml of dry THF was treated with 200 mg of CDI. After 10 min 140 mg of 2-hydroxypropionic acid were added and the mixture heated to 45° C. for 12 h. After cooling the mixture was concentrated in vacuum and the residue chromatographed on silica gel, elution with ethyl acetate to yield 220 mg of the ester. This material was dissolved in 4 ml of THF together with 300 mg of M2 and followed by addition of 100 mg of DCC. After 16 h the mixture was filtered and the residue was concentrated in vacuum. The residue was chromatographed on silica gel, elution with chloroform:isopropanol:ammonia (7 M in methanol) 40:1:1 to yield 290 mg of the desired product. MS: 507.8 (M+2H⁺).

### Example 14

### Uptake of Conjugates

Freshly drawn heparinised blood or buffy coat preparations are used for the determination of conjugate uptake. Buffy coat preparations are preferred. They may be obtained from donor blood by simple centrifugation of whole blood (4795 g for 10 minutes). Following centrifugation, plasma is collected from the surface, after which immune cells are expressed from the donor bags along with the erythrocytes lying immediately below the leukocyte layer. This ensures high yields and a sufficient population of erythrocytes for partition. 5 ml of the resulting cell suspension are dispensed into T25 culture flasks. Substrates are added to a final concentration between 1 and 10 μM and the suspensions incubated at 37° C., in a 5% CO₂ atmosphere. For analysis of uptake kinetics, samples are drawn at 0, 2, 5, 10, 30, 60, 90, 180, or 240 min after substrate addition. For screening purposes, samples are taken at 0 and 120 minutes.

Buffers and solutions:

### PBS 73 mM NaCl, 2.7 mM KCl, 1.5 mM KH₂PO₄, 8 mM Na₂HPO₄ pH 7.4

### DPBS 137 mM NaCl, 3 mM KCl, 8 mM Na₂HPO₄, 1 mM KH₂PO₄, 1 mM CaCl₂, 0.5 mM MgCl₂, 5 mM Glucose pH 7.4

### Separation of Blood Cell Fractions—Density Gradient Centrifugation

Cell fractions were prepared using density gradient centrifugation. Mononuclear cells and polymorphonuclear cells are separated from erythrocytes essentially by layering the cell suspension on a viscous medium typically composed of a solution containing Ficoll or similar (commercial suppliers include: Lymphoprep, Axis Shield, 1031966; Lymphoflot HLA, 824010; or PMN Separation Medium Robbins Scientific 1068-00-0). The layered suspension is then centrifuged at 600 g, 20 min, after which the cell fractions and the plasma (incubation medium) fraction are removed by gentle aspiration, washed twice in PBS buffer, followed by estimation of the cell number and pellet volume.

### Analysis

Uptake of compounds is monitored using chromatographic analysis (LC/MS) with mass selective detection. Uptake is also normalized to the amount of cells based on both estimated volume and total protein. To obtain these data, cell preparations are lysed in water and the debris sedimented at 16100 g, 10 min. The supernatant is recovered and sub-sampled for protein and DNA content. Protein in the supernatant is precipitated by bringing the solution to 100% v/v ethanol and centrifuging again at 16100 g, 10 min. Compound uptake is normalized according to cytoplasmic volume of cells in order to obtain the average concentration in the cells. Cell volume is estimated by correlation of DNA, protein or haem content of lysed cell aliquots to cell number and packed volume prior to lysis.

TABLE 1
Ratio of the concentration of compounds outside and inside cells

| | Ratio internal over external |
|---|---|
| Compound | concentration |
| Macrolide for compounds 5 and 8 alone | ++ |
| Compound 8 | ++ |
| Drug component of 8 (without macrolide) | + |
| Compound 5 | +++ |
| Drug component of 5 (without macrolide) | + |

Key: − = excluded from blood cells;
+ = equal or similar;
++ = >5-fold enhancement in blood cells;
+++ = >10-fold enhancement in blood cells.

### Example 15

### Efficacy in Animal Models

Efficacy of drugs in suppressing arthritis may be determined using animal models. The collagen-induced arthritis in rodents is a well established experimental model of rheumatoid arthritis and considered appropriate to test the efficacy of anti-inflammatory drugs. The model is performed by immunising animals with exogenous collagen (e.g. bovine or chick) and administering substances following the appearance of disease. Disease may be boosted to increase response. The data reported herein were generated using the murine model in DBA J1 mice. At day zero a collagen suspension in complete Freund's adjuvant was injected s.c. at the tail base. At day 20, collagen in incomplete Freund's adjuvant was injected at a nearby position on the tail base. When arthritis developed, animals were randomly assigned to treatment groups. Signs monitored included weight, paw score, paw thickness and overall condition. Animals were observed and treated for 10 days. Macrolide conjugates were formulated as solutions in 1.5% citrate, 6% fructose in water. The results are shown in the following table 2.

TABLE 2
Efficacy of drugs in suppressing arthiritis

| | arthitic score* |
|---|---|
| Compound 8 | + |
| Active moiety of compound 8 | ++ |
| Compound 5 | − |
| Active moiety of compound 5 | ++++ |
| Vehicle | ++++ |

*all joints were graded according to a scoring system based on the following:
−, healthy paw;
+, one joint inflamed;
++, two types of joint inflamed (e.g. tarsal and digit);
+++, inflammation of three joints in two separate parts of the paw;
++++, inflammation of whole paw.

In a further experiment Arthritis was induced using bovine collagen as described above. Animals were treated with compounds p.o. once daily following positive development of disease. Comparison with control animals (untreated) was made at 9 days after treatment. The results are shown in the following table 3.

TABLE 3
Arthritic score in animals treated
with various macrolide conjugates.

| | relative score at day 9 |
|---|---|
| | after onset of arthritis |
| Vehicle | 100% |
| Active moiety alone | |
| dose: 132 μmol/kg | 90% |
| Compound 2 | |
| dose: 66 μmol/kg | 44% |
| Compound 4 | |
| dose: 66 μmol/kg | 76% |
| dose: 132 μmol/kg | 33% |
| Compound 9 | |
| dose: 66 μol/kg | 67% |
| dose: 132 μmol/kg | 14% |

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### SCIENTIFIC CITATIONS

1. Ianaro et al., 2000, Anti-inflammatory activity of Macrolide Antibiotics. J. Pharmacol. Ex. Therapeutics. 292: 156-161.
2. Labro M T and Abdelghaffar H, 2001, Immunomodulation by macrolide antibiotics. J. Chemother. February; 13(1): 3-8. Review.
3. Labro M T, 1998, Anti-inflammatory activity of macrolides: a new therapeutic potential? J. Antimicrobial Chemother. 41, Suppl., 37-46.
4. Laufer S, Tries S, Augustin J, Dannhardt G. Pharmacological profile of a new pyrrolizine derivative inhibiting the enzymes cyclo-oxygenase and 5-lipoxygenase. Arzneimittelforschung 1994; 44: 629-36.
5. Laufer S, Tries S, Augustin J, Elsässer R, Albrecht W, Guserle R, et al. Acute and chronic anti-inflammatory properties of [2,2-dimethyl-6-(4-chlorophenyl)-7-phenyl-2,3-dihydro-1H-pyrrolizine-5-yl]-acetic acid. Arzneimittelforschung 1995; 45: 27-32.
6. Laufer S, Tries S, Augustin I, Elsässer R, Algate D R, Atterson P R, Munt P L (1994) Gastrointestinal Tolerance of [2,2-Dimethyl-6-(4-chlorophenyl)-7-phenyl-2,3-dihydro-1H-pyrrolizine-5 yl]-acetic Acid in the Rat. Arzneim.-Forsch./Drug Res. 44 (II): 1329-1333.
7. Tries S and Laufer S (2001) The pharmacological profile of ML3000: A new pyrrolizine derivative inhibiting the enzymes cyclo-oxygenase and 5-lipoxygenase. Inflammopharmacology 9: 113-124.
8. Wallace J L, Carter L, McKnight E., Tries S, Laufer S (1994), ML 3000 reduces gastric prostaglandin synthesis without causing mucosal injury, Eur. J. Pharmacol. 271, 1994, 525-531.
9. Laufer S, Augustin J, Danhardt G, Kiefer W, A novel class of potent dual inhibitors of both cyclooxygenase and 5-lipoxygenase, J. Med. Chem. 1994, 37, 1894-1897.
10. Laufer S et al., Synthesis and evaluation of a novel series of pyrrolizine derivatives as dual cyclooxygenase and 5-lipoxygenase inhibitors, Arch. Pharm. Med. Chem. 330, 307-312 (1997).
11. Drugs of the Future 1995, 20 (10): 1007-1009 mL 3000 Antiinflammatory Cyclooxygenase and 5-Lipoxygenase Inhibitor

1. A macrolide conjugate of formula I 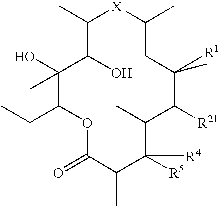
wherein
R¹ is hydroxy or C₁-C₄-alkoxy or
R¹ and R⁴ together with the carbon atoms to which they are attached form a tetrahydrofurane ring,
R²¹ is 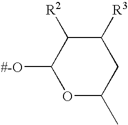
one of radicals R² and R³ is OR⁹ and the other is NR⁶R⁷;
R⁴ is OH, OR⁹ or 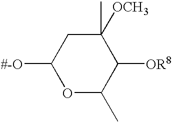
R⁵ is H or
R⁴ and R⁵ together with the carbon atom to which they are attached form a carbonyl group;
R⁶ and R⁷ which may be the same or different are C₁-C₄-alkyl or R⁹O—C₁-C₄-alkyl;
R⁸ is H or R¹⁰;
R⁹ is H or R¹⁰;
R¹⁰ is 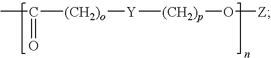
X is NR¹¹CH₂, CH₂NR¹¹, C═O or C═NOR²⁰
R¹¹ is H or C₁-C₄-alkyl;
R²⁰ is H, R¹⁰ or —(CH₂)ₖ—Y—(CH₂)_{I}—Y—(CH₂)ₘ—CH₃;
Y is O or a bond;
k is 1 or 2;
l is 1, 2 or 3;
m is 0, 1 or 2;
n is 0 or 1;
o is 1, 2, or 3;
p is 1, 2, or 3;
Z is 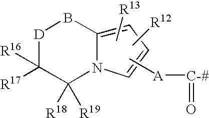
wherein
R¹² and R¹³ which may be the same or different are selected from:
phenyl which is optionally substituted with 1 or 2 halogen, hydroxy, C₁-C₄-alkoxy, phenoxy, C₁-C₄-alkyl or CF₃,
a 5- or 6-membered aromatic heterocyclic group containing 1, 2 or 3 heteroatoms selected from O, N, or S and which may be substituted with 1 or 2 halogen, C₁-C₄-alkyl or CF₃,
a benzofused 5- or 6-membered aromatic heterocyclic group containing 1, 2 or 3 heteroatoms selected from O, N, or S and which may be substituted with 1 or 2 halogen, C₁-C₄-alkyl or CF₃;
A is a bond or C₁-C₈-alkylene which can optionally be substituted by hydroxyl or
C₁-C₄-alkoxy;
B is CR¹⁴R¹⁵ or C═O;
R¹⁴ and R¹⁵ which may be the same or different are H or C₁-C₄-alkyl or one of radicals R¹⁴ and R¹⁵ is H, C₁-C₄-alkyl, hydroxy-C₁-C₄-alkyl or C₁-C₄-alkoxy-C₁-C₄-alkyl and the other is OH, C₁-C₄-alkoxy or C₁-C₄-alkylcarbonyloxy;
D is a bond between B and the carbon atom carrying R¹⁶ and R¹⁷ or is CH₂;
R¹⁶ and R¹⁷ which may be the same or different are H, C₁-C₄-alkyl, hydroxy-C₁-C₄-alkyl or C₁-C₄-alkoxy-C₁-C₄-alkyl;
R¹⁸ and R¹⁹ which may be the same or different are H or C₁-C₄-alkyl or
two of radicals R¹⁶, R¹⁷, R¹⁸ and R¹⁹ form a double bond and the two others are H or C₁-C₄-alkyl,
and the pharmaceutically acceptable salts, solvates, hydrates and stereochemical isomers thereof.

2. The macrolide conjugate according to claim 1, wherein R² is OR⁹ and R³ is NR⁶R⁷.

3. The macrolide conjugate according to claim 2, wherein R² is OR¹⁰ and R³ is NR⁶R⁷, wherein R⁶ and R⁷ are C₁-C₄-alkyl.

4. The macrolide conjugate according to claim 2, wherein R² is OH and R³ is NR⁶R⁷, wherein R⁶ is C₁-C₄-alkyl and R⁷ is R¹⁰O—C₁-C₄-alkyl.

5. The macrolide conjugate according to claim 1, wherein R² is NR⁶ R⁷ and R³ is OR⁹.

6. The macrolide conjugate according to claim 5, wherein R⁶ and R⁷ which may be the same or different are C₁-C₄-alkyl and R³ is OR¹⁰.

7. The macrolide conjugate according to claim 5, wherein R⁶ is C₁-C₄-alkyl, R⁷ is R¹⁰O—C₁-C₄-alkyl and R³ is OH.

8. The macrolide conjugate according to claim 1, wherein R⁴ is OH or

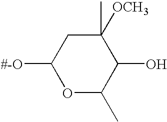

9. The macrolide conjugate according to claim 1, wherein R⁴ and R⁵ together with the carbon atom to which they are attached form a carbonyl group.

10. The macrolide conjugate according to claim 1, wherein
X is NR¹¹CH₂,
C═NO(CH₂)ₖY(CH₂)ₗY(CH₂)ₘ—CH₃ or
C═NOR¹⁰,
wherein
R¹¹ is C₁-C₄-alkyl and R¹⁰, Y, k, l and m are as defined in claim 1.

11. The macrolide conjugate according to claim 1, wherein
R¹⁰ is Z or —CO—(CH₂)ₒ—Y—(CH₂)ₚ—O-Z, wherein Y is a bond and Z, o and p are as defined in claim 1.

12. The macrolide conjugate according to claim 1 having formula Ia 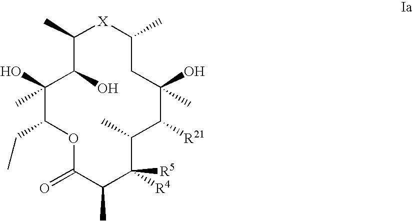
wherein R⁴, R⁵, R²¹ and X are as defined in claim 1.

13. The macrolide conjugate according to claim 12 selected from the formulae Iaa to Iaf: 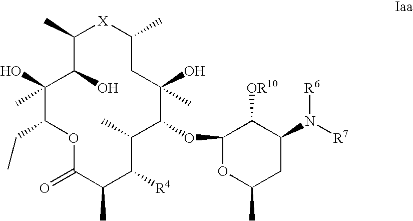
wherein R⁶ and R⁷ which may be the same or different are C₁-C₄-alkyl; 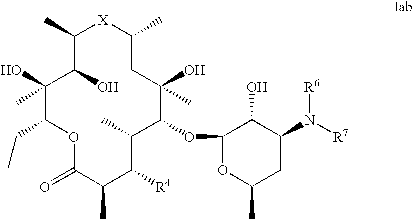
wherein R⁶ is C₁-C₄-alkyl and R⁷ is hydroxy-C₁-C₄-alkyl or R¹⁰O—C₁-C₄-alkyl; 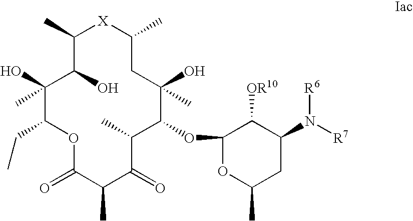
wherein R⁶ and R⁷ which may be the same or different are C₁-C₄-alkyl; 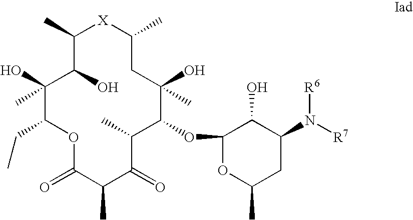
wherein R⁶ is C₁-C₄-alkyl and R⁷ is hydroxy-C₁-C₄-alkyl or R¹⁰O—C₁-C₄-alkyl; 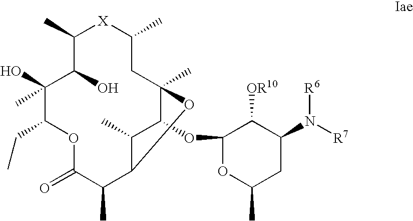
wherein R⁶ and R⁷ which may be the same or different are C₁-C₄-alkyl; 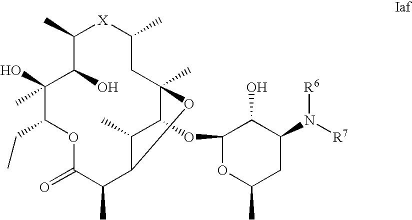
wherein R⁶ is C₁-C₄-alkyl and R⁷ is hydroxy-C₁-C₄-alkyl or R¹⁰O—C₁-C₄-alkyl; and wherein in formulae Iaa to Iaf R⁴ is hydroxy or 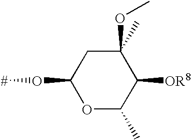
and X, R⁸ and R¹⁰ are as defined above.

14. The macrolide conjugate according to claim 13, wherein in formula Iaa to Iaf X is NR¹¹CH₂.

15. The macrolide conjugate according to claim 1, wherein R¹² and R¹³ which may be the same or different are phenyl, thienyl, furyl, pyrrolyl, imidazolyl, thiadiazolyl, oxazolyl, pyridinyl, pyrimidyl, benzofuryl, quinolyl, or indolyl and may be substituted with one or two halogens or CF₃.

16. The macrolide conjugate according to claim 15, wherein R¹² and R¹³ which may be the same or different are phenyl, halogen-substituted phenyl, thienyl, halogen-substituted thienyl or benzofuryl.

17. The macrolide conjugate according to claim 15, wherein R¹⁴, R¹⁵, R¹⁸ and R¹⁹ are H and R¹⁶ and R¹⁷ are H or C₁-C₄-alkyl.

18. The macrolide conjugate according to claim 1, wherein Z is 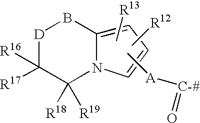
wherein A is a bond or C₁-C₈-alkylene,
R¹² is phenyl, halogen-substituted phenyl, thienyl, halogen-substituted thienyl, or benzofuryl;
R¹³ is phenyl;
R¹⁶ and R¹⁷ are H or C₁-C₄-alkyl; and
R¹⁸ and R¹⁹ are H.

19. The macrolide conjugate according to claim 18, wherein A is CH₂ and R¹² is chlorophenyl, chlorothienyl or benzofuryl.

20. The macrolide conjugate according to claim 19, wherein R¹² is 4-chlorophenyl, 5-chlorothien-2-yl or benzofur-2-yl.

21. The macrolide conjugate according to claim 20, wherein Z is

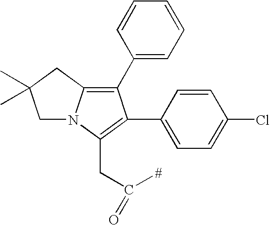

22. A pharmaceutical composition comprising a macrolide conjugate as defined in claim 1, together with a pharmaceutically acceptable excipient.

23. The pharmaceutical composition according to claim 22 in the form of a parenteral or topical formulation.

24. (canceled)

25. A method of treating rheumatic type disorders, which comprises administering to an individual in need of such treatment an effective amount of a macrolide conjugate according to claim 1.
